(19) United States
(12) Patent Application Publication
(10) Pub. No.: US 2005/0048066 A1
(43) Pub. Date: Mar. 3, 2005

(54) Identification of potential immunodominant acetylcholine receptor alpha subunit

(76) Inventors: Shrikant Deshpande, Fremont, CA (US); Edward Spack, Mountain View, CA (US); Nancy Wehner, Fremont, CA (US); Subhashini Arimilli, Fremont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/240,035

(22) PCT Filed: Mar. 30, 2001
(86) PCT No.: PCT/US01/10450

Related U.S. Application Data
(60) Provisional application No. 60/193,745, filed on Mar. 31, 2000.

(51) Int. Cl. A61K 39/00

(52) U.S. Cl. 424/185.1

(57) ABSTRACT

The present invention is directed to the treatment of autoimmune diseases, in particular of Myasthenia Gravis. This invention provides novel autoimmune dominant peptides derived from the acetylcholine receptor, as well as methods for preparing the peptides. The present invention further provides complexes comprising these peptides associated with an appropriate major histocompatibility complex (MHC) molecule and methods for making these complexes. The complexes of the present invention can be used therapeutically or prophylactically for treating Myasthenia Gravis.

### BACKGROUND OF THE INVENTION

Autoimmune diseases are a particularly important class of deleterious immune responses and constitute a major health problem in the United States, affecting over 9 million people. In autoimmune diseases, self-tolerance is lost and the immune system attacks self-tissue as if it were a foreign target. Lacking cures, these diseases are generally chronic and in many instances fatal.

A crude approach to treating autoimmune diseases is general immunosuppression. This has the obvious disadvantage of crippling the ability of the subject to respond to real foreign materials to which it needs to mount an immune response. An only slightly more sophisticated approach relies on removal of the immune complexes involving the target tissue. This also has adverse side effects, and is difficult to accomplish. Aside from immunosuppressant agents, which have deleterious side effects, there are few effective long term treatments for any autoimmune disease. There are over 30 different organ specific and systemic forms of autoimmunity with varying incidence rates. Those with relatively large patient populations, such as rheumatoid arthritis, type I diabetes, and multiple sclerosis, are for obvious economic reasons more frequently targeted for development of novel therapeutics.

A growing understanding of the role T lymphocytes play in the maintenance of tolerance and the pathology of autoimmunity sparked development of a variety of antigen-specific therapeutics intended to suppress or eliminate autoreactive T cells while preserving protective immunity. These strategies depend on identification of the autoantigens and autoantigen peptide epitopes involved in the autoimmune disease. For many autoimmune diseases, including those with the largest patient populations, this information is unknown, incomplete, or controversial.

Myasthenia Gravis (MG) is an autoimmune disease that affects the neuromuscular junction and can be life-threatening. The patient population size is variably estimated at 25,000-100,000 patients (Drachman (1994) N. Eng. J. Med. 330:1797-1810; MG Foundation, 1997), and increased case numbers are anticipated as the mean population age increases. MG is characterized by auto-antibodies to the acetylcholine receptor (AChR). In vivo animal models and immunosuppressive drugs indicate that generation of these auto-antibodies is dependent on CD4+T cells. In MG, autoantibodies bind to the acetylcholine receptor (AChR) on the muscle membrane at the neuromuscular junction, causing endocytosis of the AChR and complement-mediated damage. The loss of AChR reduces the efficiency of muscle function, with symptoms ranging from fatigue to respiratory failure.

MG offers several advantages for the development and clinical testing of antigen-specific therapeutics, including the existence of a well characterized autoantigen, AChR. AChR is widely acknowledged as the target of autoreactive T and B cells, unlike diseases such as multiple sclerosis where multiple myelin proteins (including myelin basic protein, proteolipid protein, myelin oligodendrocyte glycoprotein, myelin associated glycoprotein, αB-crystallin, CNPase, and heat shock proteins) may be involved. In addition, definitive clinical parameters are available. For example, the tension test, which measures the temporary decrease in muscle impairment due to blocking of acetylcholinesterase, and the detection of anti-AChR antibodies are diagnostic tools for MG. The disease is associated with HLA-DR3 in early onset patients and HLA-DR2 in late onset. Furthermore, muscle fimction is readily measured by electromyography, and anti-AChR levels may be followed by ELISA and other relatively simple assays, so a clinical trial in MG benefits from simple, definitive surrogate markers of disease activity. In spite of these advantages, few therapeutic strategies have been tested in MG.

The greatest difficulty in developing antigen-specific therapeutics in MG is the current lack of consensus on the identity of the immunodominant AChR epitope(s).

AChR is a pentameric ion channel composed of α2βεδ in adult innervated muscle and α2βγδ in embryonic and denervated muscle and in myoid cells of the thymus. The AChRα subunit bears the binding site for acetylcholine as well as the major immunogenic region (MIR) at 67-76, a dominant site of autoantibody binding (Tzartos et al. (1989) Proc. Natl. Acad. Sci. USA 85:2899-2903). Although there are many reports of dominant AChR T cell epitopes in the literature, many of these studies are not corroborated and there is no clear consensus (reviewed in, e.g., Manfredi et al. (1992) J. Lab. Clin. Med. 1:13-21; Hawke et al. (1996) Immunol. Today 17:307-311). Most of these studies failed to test HLA-DR restrictions. Furthermore, several studies based the identification of immunodominant peptides on the generation of T cell lines reactive to synthetic AChR peptides, but in many cases these T cells failed to react to native AChR (Matsuo et al. (1995) J. Immunol. 155:3683-3692; Hawke et al., supra). It is thus unclear if these T cells are artifacts of the assay system.

Previous studies have also examined the response of MG patient peripheral blood mononuclear cells to a set of AChR peptides (see, e.g., Harcourt et al. (1988) J. Clin. Invest. 82:1894; and Newsom-Davis et al. (1989) J. Autoimmun. 2:101). The studies, however, tested either an incomplete set of AChR peptides or a set of peptides in which the overlap of consecutive peptides was limited to a few amino acids, possibly missing the relevant epitope. In other cases, the HLA-DR restriction of the T cell response was not determined. Other studies have identified immunodominant peptides that bind HLA-DR alleles and may have significance for MG, but the T cell reactivity to these has not been confirmed. Despite these advances, the art lacks consensus on HLA-DR associated immunodominant AChR peptides.

The current therapies for MG are not specific, and in many cases have deleterious side effects. For example, plasmaphoresis to remove antibodies is an expensive approach, and when terminated the titer can quickly return and exceed the pre-treatment level. Steroids and other immunosuppressants (e.g., ACTH, azathioprine and cyclosporin A) are sometimes administered, but are often accompanied by nephrotoxicity, hypertension, or other health risks. Thymectomy is effective in many cases, but also often fails, may require up to five years to establish effect, and is contraindicated in the very young and the elderly.

There is therefore a need in the art for methods for treating autoimmune disease, in particular MG, efficiently, without major side affects and without affecting the entire immune system. This invention addresses these and other needs.

### SUMMARY OF THE INVENTION

The present invention is directed to compositions that can be used to inhibit those aspects of the immune system responsible for the autoimmune response in myasthenia gravis. The invention compositions are designed to target helper T cells which recognize a particular antigen in association with an MHC component. The invention compositions bind T cells and cause non-responsiveness in target T cells, providing a specific therapy with fewer side effects that previous therapies.

The invention provides identification of AChR subunit peptides with relatively high affinity for HLA-DR2 and HLA-DR3, and which may represent immunodominant T cell epitopes in MG patients with these haplotypes. These peptides can then be used to induce nonresponsiveness in target T cells and thus treat MG.

In one embodiment, the invention provides the peptides. In other embodiments, the peptides can be used in pharmaceutical compositions to treat MG, or can be complexed to an MHC molecule and then used in a pharmaceutical composition. In yet another embodiment, the invention provides a method of treating MG.

### BRIEF DESCRIPTION OF THE DRAWINGS

FIG. 1 shows the position of overlapping AChR peptides tested in the DELFIA assay of affinity for HLA-DR2 and HLA-DR3.

FIG. 2 lists the IC₅₀ values of each AChR peptide for binding to HLA-DR2 and HLA-DR3. IC₅₀ values above 100,000 nM are indicated by the symbol “>”.

FIG. 3 shows an histogram of AChRα peptide 1/values, illustrating the relative affinity of the peptides for HLA-DR2 and HLA-DR3.

FIG. 4 shows possible DRB1*1501 motif alignments of AChRα peptides with high affinity for HLA-DR2. The best fit of AChRα peptide sequences to published HLA-DR2 motifs is indicated along with the IC₅₀ value.

FIG. 5 shows possible DRB 1*0301 motif alignments of AChRα peptides with high affinity for HLA-DR3. The best fit of AChRα peptide sequences to published HLA-DR3 motifs is indicated along with the IC₅₀ value.

FIG. 6 lists candidate immunodominant AChR peptides for HLA-DR2 and HLA-DR3. Those with IC₅₀ values ≦10,000 nM are listed.

FIG. 7 shows ELISPOT assay results for PBMCs from normal, healthy individual samples. A reactive index of 2 or more for a particular antigen is considered to be positive.

FIG. 8 shows ELISPOT assay results for PBMCs from MG patient samples. T cell reactivity is identified as positive if the reactive index for a particular antigen is greater than two.

FIG. 9 shows a comparison of the percentage of normal healthy individuals (“normals”) or MG patients (“patients”) that show a positive reactivity to different AChR peptides.

FIG. 10 shows a comparison of the percentage of DR2+ normal healthy individuals (“normals”) or DR2+ MG patients (“patients”) showing reactivity to AChR peptides.

### DESCRIPTION OF THE SPECIFIC EMBODIMENTS

### I. Introduction

The present invention provides peptides which can be used to modulate T cell finction. For instance, the peptides can be bound to an MHC molecule and used to inhibit a deleterious T cell-mediated immune response, in particular in myasthenia gravis. In addition the peptides themselves can be used to induce non-responsiveness and can be used to treat disease.

The peptides and the MHC components with which they can be complexed are each described separately below, followed by a description of the methods by which the peptides and the complexes can be prepared, evaluated and employed. General methods suitable for making and using MHC:peptide complexes of the invention are disclosed in U.S. Pat. No. 5,468,481 and in the PCT Patent Application WO 96/40944.

In particular, this invention provides novel acetylcholine receptor (AChR) peptides, MHC Class II polypeptides, and MHC Class II:AChR peptide complexes. The invention provides isolated (from natural sources), synthetic, and recombinantly generated forms of AChR peptides and MHC Class II polypeptides. These peptides and proteins can be recombinantly expressed in vitro or in vivo. The peptides, polypeptides and complexes of the invention can be made and isolated using any method known in the art, and the invention provides a few exemplary means for generating such proteins. In addition, means to make MHC Class II:peptide complexes are taught in, e.g., U.S. Pat. Nos. 5,194,425, issued Mar. 16, 1993; 5,130,297, issued Jul. 14, 1992; 5,284,935, issued Feb. 8, 1994; 5,260,422, issued Nov. 9, 1993; and 5,468,481, issued Nov. 21, 1995; and in PCT Patent Application No. WO 96/40944.

Techniques for nucleic acid manipulation and recombinant expression of genes encoding the AChR peptides, the MHC Class II molecules and the AChR peptides:MHC Class II complexes of the invention are described in the scientific and patent literature (see, e.g., Sambrook et al., Molecular Cloning: A Laboratory Manual (2nd Ed.), Vols. 1-3, Cold Spring Harbor Laboratory, (1989); Ausubel et al., Current Protocols in Molecular Biology, John Wiley & Sons, Inc., New York (1997); and Tijssen et al., Laboratory Techniques in Biochemistry and Molecular Biology: Hybridization With Nucleic Acid Probes, Part I. Theory and Nucleic Acid Preparation, Elsevier, N.Y. (1993)). Mutations can be introduced into a nucleic acid by a variety of conventional techniques, well known to those of skill in the art. For example, one rapid method to perform site-directed mutagenesis efficiently is the overlap extension polymerase chain reaction (Urban (1997) Nucleic Acids Res. 25:2227-2228). Sequencing methods to verify the sequence of the nucleic acid of interest typically use dideoxy sequencing (Sequenase, U.S. Biochemical), however, other kits and methods are available and well known to those of skill in the art. A variety of in vivo expression systems and techniques for transforming prokaryotic and eukaryotic cells can be used and are well known to those of skill in the art (see, e.g., Weising (1988) Ann. Rev. Genet. 22:421-477; Sambrook et al.; and Tijssen et al., both supra).

AChR peptides, MHC Class II molecules and MHC Class II:peptide complexes of the invention can also be synthesized, whole or in part, using chemical methods well known in the art (see, e.g., Caruthers (1980) Nucleic Acids Res. Symp. Ser. 215-223; Horn (1980) Nucleic Acids Res. Symp. Ser. 225-232; and Banga, Therapeutic Peptides and Proteins, Formulation, Processing and Delivery Systems Technomic Publishing Co., Lancaster, Pa. (1995)). For example, peptide synthesis can be performed using various solid-phase techniques (see, e.g., Roberge (1995) Science 269:202; and Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

### II. Definitions

As used herein, a “peptide” or “oligopeptide” is a series of residues, typically L-amino acids, connected one to the other typically by peptide bonds between the alpha-amino and carbonyl groups of adjacent amino acids. The length of the peptides is not critical to the invention so long as the correct epitopes are maintained. The peptides are typically less than about 30 residues in length and usually consist of between about 10 and about 25 residues, preferably 14 to 20 residues.

An “AChR oligopeptide” is one having a sequence derived from a portion of AChR that specifically binds an MHC molecule and induces an immune response in T cells associated with myasthenia gravis. An oligopeptide can comprise a sequence from AChR (e.g., residues 7-22 or 3649), or one substantially identical to it. The term also encompasses various analogs of such sequences, as described below.

The term “residue” refers to an amino acid (D or L) or amino acid mimetic incorporated in an oligopeptide by an amide bond or amide bond mimetic. An amide bond mimetic of the invention includes peptide backbone modifications well known to those skilled in the art.

“Amino acid mimetic” as used here is a moiety other than a naturally occurring amino acid that conformationally and functionally serves as a substitute for an amino acid in a peptide of the present invention. Such a moiety serves as a substitute for an amino acid residue if it does not substantially interfere with the ability of the peptide to bind to AChR. Amino acid rnimetics may include non-protein amino acids, such as β-γ-δ-amino acids, β-γ-δ-imino acids (such as piperidine-4-carboxylic acid) as well as many derivatives of L-α-amino acids. A number of suitable amino acid mimetics are known to the skilled artisan; they include cyclohexylalanine, 3-cyclohexylpropionic acid, L-adamantyl alanine, adamantylacetic acid and the like. Peptide mimetics suitable for peptides of the present invention are discussed by Morgan and Gainor (1989) Ann. Repts. Med. Chem. 24:243-252.

Two polypeptides are said to be “identical” if the sequence of amino acid residues in the two sequences is the same when aligned for maximum correspondence.

“Percentage of sequence identity” is determined by comparing two optimally aligned sequences over a comparison window, wherein the portion of the polynucleotide or amino acid sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of amino acid or nucleic acid sequences for these purposes normally means sequence identity of at least 60%. Preferred percent identity of polypeptides or polynucleotides can be any integer from 60% to 100%. More preferred embodiments include at least 80%, 85%, 90%, 95%, or 99%. Polypeptides which are “substantially similar” share sequences as noted above except that residue positions which are not identical may differ by conservative amino acid changes. “Conservative amino acid substitutions” refer to the interchangeability of residues having similar side chains. For example, a group of amino acids having aliphatic side chains is glycine, alanine, valine, leucine, and isoleucine; a group of amino acids having aliphatic- hydroxyl side chains is serine and threonine; a group of amino acids having amide- containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

Optimal alignment of sequences for comparison may be conducted by the local identity algorithm of Smith and Waterman (1981) Add. APL. Math. 2:482, by the identity alignment algorithm of Needleman and Wunsch (1970) J. Mol. Biol. 48:443, by the search for similarity method of Pearson and Lipman (1988) Proc. Natl. Acad. Sci. (U.S.A.) 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

A preferred example of algorithms that are suitable for determining percent sequence identity and sequence similarity are the BLAST and BLAST 2.0 algorithms, which are described in Altschul et al. (1977) Nuc. Acids Res. 25:3389-3402 and Altschul et al. (1990) J. Mol. Biol. 215:403-410, respectively. BLAST and BLAST 2.0 are used, with the parameters described herein, to determine percent sequence identity for the nucleic acid and amino acid sequences of the invention. Software for performing BLAST analyses is publicly available through the National Center for Biotechnology Information (http://www.ncbi.nlm.nih.gov/). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The phrase “isolated” refers to material which is substantially or essentially free from components which normally accompany it as found in its native state.

The terms “immunodominant” or “immunodominant peptides” when used herein refer to those peptides or peptide epitopes that serve as the primary target for an immune response. Immunodominant peptides are typically those that evoke antibodies in larger quantifies and with higher binding affinities than do other available epitopes. Identification of these is important because they are the ones which should be targeted to fight autoimmune diseases. Immunodominance is the property of certain epitopes within a complex antigen (or certain residues within an epitope) that makes them the most critical for immunogenicity or antigenicity.

The term “isolated MHC component” as used herein refers to an MHC glycoprotein or an effective portion of an MHC glycoprotein (i.e., one comprising an antigen binding site or sites and sequences necessary for recognition by the appropriate T cell receptor) which is in other than its native state, for example, not associated with the cell membrane of a cell that normally expresses MHC. As described in detail below, the MHC component may be recombinantly produced, solubilized from the appropriate cell source or associated with a liposome. For human MHC molecules, human lymphoblastoid cells are particularly preferred.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under-expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “operably linked” refers to a finctional linkage between a nucleic acid expression control sequence (such as a promoter, or array of transcription factor binding sites) and a second nucleic acid sequence, wherein the expression control sequence directs transcription of the nucleic acid corresponding to the second sequence.

### III. The MHC Component

The glycoproteins encoded by the MHC have been extensively studied in both the human and murine systems. The MHC gene complex is termed H-2 complex in mice and HLA complex in humans. The MHC glycoproteins have been classified as Class I glycoproteins, found on the surfaces of all cells and primarily recognized by cytotoxic T cells, and Class II, which are found on the surfaces of several cells, including accessory cells such as macrophages, and are involved in presentation of antigens to helper T cells. Some of the histocompatibility proteins have been isolated and characterized. For a general review of MHC glycoprotein structure and function, see Paul, Fundamental Immunology, 2nd Ed., Ravens Press N.Y. (1989) and Alberts et al., Molecular Biology of the Cell, 2nd Ed., Garland Publishing, Inc., N.Y. & London (1989).

MHC Class II molecules are particularly useful in the present invention. A class II MHC molecule is formed from the N-terminal domain portions of two class II chains which extend from the membrane bilayer. The N-terninal portion of one chain has two domains of homology with the alpha, and alpha₂ regions of the MHC Class I antigen sequence. The antigen binding pocket in MHC class II molecules is made up of the alpha and beta₁ domains. The binding pocket is open at both ends in Class II molecules so it can accommodate longer peptides. The three-dimensional structure of HLA-DR1 has been described (Brown et al. (1993) Nature 364:33). Cloning of the Class II genes permits manipulation of the Class II MHC binding domains for example, as described below.

The MHC glycoprotein portions of the complexes of the invention can be obtained by isolation from lymphocytes and screened for the ability to bind the desired peptide antigen. The lymphocytes are from the species of individual which will be treated with the complexes. For example, they may be isolated from human B cells from an individual suffering from the targeted autoimmune disease, which have been immortalized by transformation with a replication deficient Epstein-Barr virus, utilizing techniques known in the art.

Methods for purifying Class II histocompatibility proteins are well known in the art. They can be isolated from a multiplicity of cells using a variety of techniques. For instance, the glycoproteins can be solubilized by treatment with protease, by treatment with 3M KCl, or by treatment with a detergent. In a preferred method, detergent extraction of Class II protein from lymphocytes followed by affinity purification is used (see, e.g., Turkewitz, et al. (1983) Molecular Immunology 20:1139-1147). Various methods have been developed to produce desirable MHC Class II histocompatibility heterodimers that do not present endogenous antigens (Stern and Wiley (1992) Cell 68:465-77; Ljunggren et al. (1990) Nature 346:476-80; and Schumacher et al. (1990) Cell 62:563-67) that can be loaded with a peptide of choice.

Alternatively, the MHC component may be recombinantly expressed using techniques well known to those skilled in the art. The amino acid sequence of each of a number of Class II proteins are known, and the genes or cDNAs have been cloned. Thus, these nucleic acids can be used to express MHC polypeptides. If a desired MHC gene or cDNA is not available, cloning methods known to those skilled in the art may be used to isolate the genes. One such method that can be used is to purify the desired MHC polypeptide, obtain a partial amino acid sequence, synthesize a nucleotide probe based on the amino acid sequence, and use the probe to identify clones that harbor the desired gene from a cDNA or genomic library. The MHC polypeptides can be expressed from cloned nucleotide sequences that encode the MHC polypeptides by operably linking the truncated or fuIll-length nucleic acids to signals that direct gene expression in a desired host. A variety of suitable host are available and known to those of skill in the art.

The MHC polypeptides can then be expressed intracellularly or can be secreted from the cell using methods known to those skilled in the art.

The nucleotide sequences used to transfect the host cells can be modified according to standard techniques to yield MHC polypeptides with a variety of desired properties. Many techniques are well known to those skilled in the art. For example, the MHC polypeptides can vary from the naturally-occurring sequence at the primary structure level by amino acid insertions, substitutions, deletions, and the like. Protein fusions may also be utilized that may confer new activities or combinations of activities to the MHC polypeptide. These modifications can be used in a number of combinations to produce the final modified MHC polypeptide chain.

Amino acid sequence variants can be prepared with various objectives in mind, including facilitating purification and preparation of the recombinant polypeptide. The modified polypeptides are also useful for modifying therapeutic half-life, improving therapeutic efficacy, and lessening the severity or occurrence of side effects during therapeutic use. The amino acid sequence variants are usually predetermined variants not found in nature but exhibit the same peptide-binding and T-cell binding activity as native-sequence MHC. For instance, polypeptide fragments comprising only a portion (usually at least about 60-80%, typically 90-95%) of the primary structure may be produced. In certain preferred embodiments, the MHC polypeptides consist essentially of either the α₁ or β₁ domain from the full-length polypeptide. Such fragments typically comprise between about 50 and about 100 amino acids, preferably between about 60 and about 90, more preferably between about 70 and about 80. Alternatively, synthetic methods may be used to prepare polypeptides (see, e.g., Merrifield (1986) Science 232:341-347; Atherton et al., Solid Phase Peptide Synthesis: A Practical Approach, IRL Press, Oxford). The composition of the synthetic peptides may be confirmed by amino acid analysis or sequencing (e.g., the Edman degradation procedure; see, e.g., Creighton Proteins, Structures and Molecular Principles, Freeman and Co., New York N.Y. (1983)), or by any other suitable technique known to those of skill in the art.

In general, modifications of the sequences encoding the MHC polypeptides is readily accomplished by a variety of well-known techniques, such as site-directed mutagenesis (see, Gillman and Smith (1979) Gene 8:81-97; and Roberts et al. (1987) Nature 328:731-734). Most modifications are evaluated by routine screening in a suitable assay for the desired characteristic. For instance, the effect of various modifications on the ability of the polypeptide to bind a peptide or affect T-cell proliferation can be easily determined using the assays described below. Modifications of other properties such as redox or thermal stability, hydrophobicity, susceptibility to proteolysis, or the tendency to aggregate are all assayed according to standard techniques.

For certain applications, the MHC cDNA coding sequences are modified to delete the transmembrane domain and express the resulting soluble MHC polypeptides. Truncation of the MHC cDNA may be performed, for example, by oligonucleotide- directed deletion mutagenesis or polymerase chain reaction. Oligonucleotide-directed in vitro mutagenesis is described, for example, by Kunkel et al. (1987) Meth. Enzymol. 154:367-382 (see also, Ausubel et al., supra).

### IV. Peptides

It is believed that the presentation of antigen by the MHC glycoprotein on the surface of antigen-presenting cells (APCs) occurs subsequent to the hydrolysis of antigenic proteins into smaller peptide units. These segments are thought to be 8-18 residues in length, and contain both the agretope (recognized by the MHC molecule) and the epitope (recognized by the T cell receptor on the T-helper cell). The epitope itself is a contiguous or non-contiguous sequence of 5-6 amino acids which recognizes the antigen- specific receptor of T-helper cells. The agretope is a continuous or non-contiguous sequence which is responsible for the association of the peptide with the MHC glycoproteins.

Important peptides in MG can be identified, for example, by screening a set of overlapping AChR peptides for binding to HLA-DR2 and HLA-DR3. The binding affinity of a peptide to the MHC compound can be measured using a variety of binding assays known to those of skill in the art. An example of suitable binding assay is the europium-based competitive binding assay (see, e.g., Tompkins et al. (1993) J. Immunol. Methods 163:209-216). Additionally, the capacity of the peptides of the invention to induce a T cell response can be determined using a variety of standard methods, known to those of skill in the art, as described below.

Typically, the peptides of the invention will comprise amino acid sequences corresponding, or substantially identical or similar, to amino acid sequences of AChR. The peptides of the invention may be isolated from natural sources, synthesized or recombinantly expressed using techniques well known to those of skill in the art.

Peptides having the desired activity may be modified as necessary to provide certain desired attributes, e.g., improved pharmacological characteristics, while increasing or at least retaining substantially all of the biological activity of the unmodified peptide to bind the desired MHC molecule and induce non-responsiveness in the appropriate T cell. For instance, the peptides may be subject to various changes, such as substitutions, either conservative or non-conservative, where such changes might provide for certain advantages in their use, such as improved MHC binding. The effect of single amino acid substitutions may also be probed using D-amino acids. Such modifications can be made using well known peptide synthesis procedures, as described in, e.g., Merrifield (1986) Science 232:341-347; Barany and Merrifield, The Peptides, Gross and Meienhofer, eds. (N.Y., Academic Press), pp. 1-284 (1979); and Stewart and Young, Solid Phase peptide Synthesis, (Rockford, Ill., Pierce), 2nd Ed. (1984).

The peptides can also be modified by extending or decreasing the peptide's amino acid sequence, e.g., by the addition or deletion of amino acids. The peptides of the invention can also be modified by altering the order or composition of certain residues, it being readily appreciated that certain amino acid residues essential for biological activity, e.g., those at critical contact sites or conserved residues, may generally not be altered without an adverse effect on biological activity. The non-critical amino acids need not be limited to those naturally occurring in proteins, such as L-α-amino acids, or their D-isomers, but may include non-natural amino acids as well, such as β-γ-δ-amino acids, as well as many derivatives of L-α-amino acids.

Typically, a series of peptides with single amino acid substitutions are employed to determine the effect of electrostatic charge, hydrophobicity, etc. on binding. For instance, a series of positively charged (e.g., Lys or Arg) or negatively charged (e.g., Glu) amino acid substitutions are made along the length of the peptide revealing different patterns of sensitivity towards various MHC molecules and T cell receptors. In addition, multiple substitutions using small, relatively neutral moieties such as Ala, Gly, Pro, or similar residues may be employed. The substitutions may be homo-oligomers or hetero- oligomers. The number and types of residues which are substituted or added depend on the spacing necessary between essential contact points and certain finctional attributes which are sought (e.g., hydrophobicity versus hydrophilicity). Increased binding affinity for an MHC molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding.

Amino acid substitutions are typically of single residues. Substitutions, deletions, insertions or any combination thereof may be combined to arrive at a final peptide. Substitutional variants are those in which at least one residue of a peptide has been removed and a different residue inserted in its place. Such substitutions generally are made in accordance with the following Table 1 when it is desired to fmely modulate the characteristics of the peptide.

TABLE 1

| Original Residue | Exemplary Substitution |
|---|---|
| Ala | Ser |
| Arg | Lys, His |
| Asn | Gln |
| Asp | Glu |
| Cys | Ser |
| Gln | Asn |
| Glu | Asp |
| Gly | Pro |
| His | Lys; Arg |
| Ile | Leu; Val |
| Leu | Ile; Val |
| Lys | Arg; His |
| Met | Leu; Ile |
| Phe | Tyr; Trp |
| Ser | Thr |
| Thr | Ser |
| Trp | Tyr; Phe |
| Tyr | Trp; Phe |
| Val | Ile; Leu |

Substantial changes in function (e.g., affinity for MHC molecules or T cell receptors) are made by selecting substitutions that are less conservative than those in Table 1, i.e., selecting residues that differ more significantly in their effect on maintaining (a) the structure of the peptide backbone in the area of the substitution, for example as a sheet or helical conformation, (b) the charge or hydrophobicity of the molecule at the target site or (c) the bulk of the side chain. The substitutions which in general are expected to produce the greatest changes in peptide properties will be those in which (a) a hydrophilic residue, e.g., seryl, is substituted for (or by) a hydrophobic residue, e.g., leucyl, isoleucyl, phenylalanyl, valyl or alanyl; (b) a residue having an electropositive side chain, e.g., lysl, arginyl, or histidyl, is substituted for (or by) an electronegative residue, e.g., glutamyl or aspartyl; or (c) a residue having a bulky side chain, e.g., phenylalanine, is substituted for (or by) one not having a side chain, e.g., glycine.

The peptides may also comprise isosteres of two or more residues in the immunogenic peptide. An isostere as defined here is a sequence of two or more residues that can be substituted for a second sequence because the steric conformation of the first sequence fits a binding site specific for the second sequence. The term specifically includes peptide backbone modifications well known to those skilled in the art. Such modifications include modifications of the amide nitrogen, the a-carbon, amide carbonyl, complete replacement of the amide bond, extensions, deletions or backbone crosslinks (see, generally, Spatola, Chemistry and Biochemistry of Amino Acids, Peptides and Proteins, Vol. VII (Weinstein Ed., 1983)).

In addition, the peptides can also be modified by linkage to other molecules. For example, different N- or C-terminal groups may be introduced to alter the molecule's physical and/or chemical properties. Such alterations may be utilized to affect, for example, adhesion, stability, bio-availability, localization or detection of the molecules. For diagnostic purposes, a wide variety of labels may be linked to the terminus, which may provide, directly or indirectly, a detectable signal. Thus, the peptides of the subject invention may be modified in a variety of ways for a variety of end purposes while still retaining biological activity.

Thus, in addition to peptides derived directly from AChR amino acid sequences, a number of conformational analogs of those sequences can be used. As used herein, “conformational analogs” are molecules having spatial or polar organization sufficiently similar to the amino acid sequences of AChR to bind an MHC component. The conformational analogs of the invention may consist entirely of amino acid residues other than those found in the AChR sequence.

### V. Formation of the Complex

Soluble MHC heterodimer:peptide complexes of the present invention can be used as antagonists to therapeutically block the binding of particular T cells and antigen-presenting cells. In addition, the molecules can induce anergy, or proliferative nonreponsiveness, in targeted T cells.

The elements of the complex can be associated by standard means known in the art. The antigenic peptides can be associated noncovalently with the antigen binding site of the MHC protein by, for example, mixing the two components. Excess peptide can be removed by any of a number of standard procedures, such as ultrafiltration or dialysis. They can also be covalently bound to the antigen binding pocket using standard procedures, such as, photoaffmity labelling (see, e.g., Hall (1985) Biochemistry 24:5702-5711; Leuscher (1990) J. Biol. Chem. 265:11177-11184; Wraith (1989) Cell 59:247-255) or any other suitable mode of linkage (see, e.g., Husain (1995) Biochem. Mol. Biol. Int. 36:669-677; Traut (1995) Biochem. Cell Biol. 73:949-958; Haselgrubler (1995) Bioconjug. Chem. 6:242-248; and Carroll (1994) Bioconjug. Chem. 5:248-256).

Alternatively, the Class II: peptide complex can be designed as one contiguous recombinant polypeptide (see, e.g., PCT Publication Nos. WO 96/40944, Dec. 19, 1996; WO 96/40194, Dec. 19, 1996; and, WO 97/04360, Nov. 6, 1997). A soluble, fused MHC heterodimer:peptide molecule directed toward a desired autoimmune disease (e.g., Myasthenia Gravis) contains the antigenic peptide implicated for that autoimmune disease (e.g., an AChR peptide) properly positioned in the binding groove of the MHC molecule, without need for solubilization of MHC or exogenous loading of an independently manufactured peptide. In such a complex the MHC component and the antigenic peptide are permanently linked into a single chain configuration. These complexes eliminate inefficient and nonspecific peptide loading. Producing the claimed MHC:peptide complexes by recombinant methodology results in specific, high yield protein production, where the final product contains only the properly configured MHC:peptide complex of choice.

An oligonucleotide which encodes the peptide can be synthesized using the known codons for each amino acid. Preferably those codons which have preferred utilization in the organism which is to be used for expression are utilized in designing the oligonucleotide. Preferred codon usage for a variety of organisms and types of cells are known in the art. A suitable sequence may then be incorporated into a sequence encoding the peptides derived from the MHC component, utilizing techniques known in the art. The incorporation site will be such that, when the molecule is expressed and folded, the AChR peptide antigen will be bound to the antigen binding site of the MHC component and available as an epitope for the target T cells.

For example, an AChR peptide, disclosed here, may be connected to the N-terminal antigen binding site of a polypeptide derived from an MHC antigen associated with MG using standard recombinant DNA techniques. If the recombinant complex is to be used in mice, for example, the AChR peptide may be incorporated into a sequence encoding either the I-A^{b}-alpha or I-A^{b}-beta chain. If the AChR peptide is to be incorporated into the beta chain, for example, the oligonucleotide may be inserted as a replacement for the leader peptide. Methods of replacing sequences within polynucleotides are known in the art.

A similar protocol may be used for incorporation of the AChR peptide into a sequence encoding a peptide derived from the appropriate human HLA antigen. For example, in humans, the haplotype DR2W2 is associated with MG. Hence, the AChR peptide may be incorporated into, for example, a sequence encoding a beta-chain of a DR2 allele. The structural basis in the DR subregion for the major serological specificities DR1-9 are known, as are the sequences encoding the HLA-DR-beta chains from a number of DR haplotypes (see, e.g., Bell et al. (1987) Proc. Natl. Acad. Sci. USA 84:6234-6238).

The autoimmune antigen peptide and the MHC component may be linked via peptide linkages. However, other modes of linkage are obvious to those of skill in the art, and could include, for example, attachment via carbohydrate groups on the glycoproteins, including, e.g., the carbohydrate moieties of the alpha- and/or beta-chains.

The physical and biological properties of the soluble, fused MHC heterodimer:peptide complexes may be assessed in a number of ways. Mass spectral analysis methods such as electrospray and Matrix-Assisted Laser Desorption/Ionization Time Of Flight mass spectrometry (MALDI TOF) analysis are routinely used in the art to provide such information as molecular weight and confirm disulfide bond formation. FACs analysis can be used to determine proper folding of the single chain complex. A number of standard assays, such as Enzyme-linked Immunosorbent Assay (ELISA), can further be used to measure concentration and confirm correct folding of the soluble, fused MHC (see, e.g., WO 96/40944).

### VI. Measuring the T Cell Responses Induced by the Peptides and Complexes of the Present Invention

The peptides and AChR:MHC Class II complexes of the invention can be assayed using a variety of in vitro models well known in the art.

To activate CD4+ T cells, TCR binding by MHC Class II:peptide is not sufficient. An additional, “co-stimulatory” signal is needed. Interaction of an MHC Class II:peptide complex of the invention with TCR lacks a co-stimulatory signal. Thus, a state of antigen-specific T cell non-responsiveness is induced (Boussiotis (1994) Curr. Opin. Immunol. 6:797-807; Park (1997) Eur. J. Immunol. 27:1082-1090). This “tolerance” or “anergy” immunosuppression and re-challenge non-responsiveness can be caused by T cell clonal anergy, by a nonresponsiveness induced by immunosuppressive cytokines, or both (Schwartz (1989) Cell 57:1073-1081; Quill (1987) J. Immunol. 138:3704-3712). The degree of immunosuppression or re-challenge non-responsiveness (i.e., tolerance, anergy) can be measured by monitoring cell proliferation, cell metabolism, secretion of cytokines or lymphokines, or any form of cell activation.

Similarly, the T cell responses induced by the peptides of the invention can be measured using the above-listed methods.

T cell activation can be measured by a variety of means well known in the art. For example, T cell proliferation can be assessed, e.g., as measured by ³H-thymidine uptake or by uptake of 3-(4,5-dimethyl-thiazol-2-7′)-2,5 diphenyltetrazolium bromide (see, e.g., Liu (1997) J. Neurochem. 69:581-593). Alternatively, as T cells synthesize and secrete cytokines upon activation, the immunosuppressive efficacy of an MHC Class II:peptide complex and the T cell responses induced by the peptides of the invention can be assessed by measuring cytokine transcription, translation or secretion. Thus, a variety of cytokines and lymphokines can be quantitated, e.g., interleukins, interferons (INFs) (e.g., gamma INF), tumor necrosis factors (TNFs) (e.g., TNF beta) and the like. Methods for measuring cytokine and lymphokine production and/or secretion are well-known in the art and include, but are not limited to, immunoassays, such as enzyme-linked immunosorbent assay (ELISA).

Other assays that can be used include the ELISPOT assay. The ELISPOT assay is a modified ELISA which allows the detection of lymphokine secretion by individual T cells in response to antigen stimulation (Czerkinsky et al. (1998) J. Immunol. Methods 110:29-36). In this assay, a capture monoclonal Ab against a particular lymphokine is plated onto a filter (e.g., a nitrocellulose or PVDF filter) and Peripheral Blood Mononuclear Cells (PBMCs)+antigen are added to the wells. Upon stimulation, T cells secrete lymphokines which are captured locally by the plated anti-lymphokine antibody. At the end of the capture phase (generally 24 hours), the cells are washed away and the secreted lymphokine is detected. The secretion of a different lymphokines can be measured using the ELISPOT assays of the invention, including, e.g., IL-2, IL-4, etc. The secreted lymphokine can be detected, for example, with a second anti-lymphokine antibody. This second anti-lymphokine antibody can be labeled or coupled directly or indirectly to a label or to an easily detectable enzyme (e.g., alkaline phosphatase). Numerous labels and enzymes that can be used in the context of the present invention are available and known to those of skill in the art. At the location where a stimulated T cell secreted lymphokine, a spot is formed. This technique is more sensitive than ELISA assays and has been used by several groups to study the T cell response of MG patients (see, e.g., Link et al. (1991) J. Clin. Invest. 87:2191-2196; Sun et al. (1992) Eur. J. Immunol. 2:1553-1559; Yi et al. (1994) J. Neuroimmunol. 50:177-186; Newsom-Davis et al. (1989) J. Autoimmun. 2:101-108; Ahlberg et al. (1992) J. Immunol. 36:435-442; Link et al. (1992) J. Immunol. 36:405-414).

In addition, a modified ELISPOT assay, termed the Clonal Expansion ELISA-Spot (or CEE-SPOT) assay can also be used in the context of the present invention. In the CEE-SPOT assay, PBMCs are stimulated with antigen for an appropriate amount of time (e.g., 7 days) to promote proliferation of the antigen-specific T cells prior to re-stimulation and lymphokine capture (e.g., on day 10). This clonal expansion of reactive T cells significantly improves assay sensitivity. The production of lymphokines can be measured using this assay, including, e.g., IL-2, IL-4, etc. Typically, lymphokine is captured on a filter, preferably a PVDF filter to improve the background and intensity of the spots. Spot counting is typically carried out using videocamera imaging and computer analysis. This type of assay can also be used to identify epitopes derived from natural processing, by first stimulating T cells with the whole protein and then re-stimulating them with peptides derived from the protein.

Cell death can also be monitored, as it has been observed that prolonged incubation of resting T cells with soluble MHC Class II:pepfide complexes results in T cell apoptosis (Arimilli (1996) Immunol. Cell Biol. 74:96-104). Cell death can be measured by a variety of known procedures, e.g., by dye exclusion permeability. Apoptosis can be assessed using, e.g., cellular DNA fragmentation, observation (as with transmission electron microscopy), detection and quantitation of apoptosis-associating protein, as bcl-2, and the like (see, e.g., Arimilli (1996) supra).

The soluble MHC heterodimer:peptide complexes of the present invention can also be tested in vivo in a number of animal models of autoimmune disease, in particular in the experimental allergic myasthenia gravis model.

### VII. Formulation and Administration of the Pharmaceutical Compositions of the Invention

If the transmembrane region of the MHC subunit is included, the compositions of the invention are conveniently administered after being incorporated in lipid monolayers or bilayers. Typically liposomes are used for this purpose, but any form of lipid membrane, such as planar lipid membranes or the cell membrane of a cell (e.g., a red blood cell) may be used. The compositions are also conveniently incorporated into micelles.

Liposomes can be prepared according to standard methods, as described below. However, if the transmembrane region is deleted, the composition can be administered in a manner conventionally used for peptide-containing pharmaceuticals.

Administration is systemic and is effected by injection, preferably intravenous, thus formulations compatible with the injection route of administration may be used. Suitable formulations are found in Remington's Pharmaceutical Sciences, Mack Publishing Company, Philadelphia, Pa., 17th ed. (1985). A variety of pharmaceutical compositions comprising complexes of the present invention and pharmaceutically effective carriers can be prepared. The pharmaceutical compositions are suitable in a variety of drug delivery systems. For a brief review of present methods of drug delivery, see, Langer (1990) Science 249:1527-1533.

In preparing pharmaceutical compositions of the present invention, it is frequently desirable to modify the complexes of the present invention to alter their pharmacokinetics and biodistribution. For a general discussion of pharmacokinetics, see, Remington's Pharmaceutical Sciences, supra, Chapters 37-39. A number of methods for altering pharmacokinetics and biodistribution are known to one of ordinary skill in the art (see, e.g., Langer, supra). For instance, methods suitable for increasing serum half-life of the complexes include treatment to remove carbohydrates which are involved in the elimination of the complexes from the bloodstream. Preferably, substantially all of the carbohydrate moieties are removed by the treatment. Substantially all of the carbohydrate moieties are removed if at least about 75%, preferably about 90%, and most preferably about 99% of the carbohydrate moieties are removed. Conjugation to soluble macromolecules, such as proteins, polysaccharides, or synthetic polymers, such as polyethylene glycol, is also effective. Other methods include protection of the complexes in vesicles composed of substances such as proteins, lipids (for example, liposomes), carbohydrates, or synthetic polymers.

Common surfactants well known to one of skill in the art can be used in the present invention. Suitable surfactants include sodium laureate, sodium oleate, sodium lauryl sulfate, octaoxyethylene glycol monododecyl ether, octoxynol 9 and PLURONIC F-127® (Wyandotte Chemicals Corp.). Preferred surfactants are nonionic polyoxyethylene and polyoxypropylene detergents compatible with IV injection such as, TWEEN-80®, PLURONIC F-68®, and the like. A preferred detergent is dodecyl-β-maltoside. In addition, phospholipids, such as those described for use in the production of liposomes, may also be used for micelle formation.

Since the MHC subunits of the present invention comprise a lipophilic transmembrane region and a relatively hydrophilic extracellular domain, mixed micelles are formed in the presence of common surfactants or phospholipids and the subunits. The mixed micelles of the present invention may comprise any combination of the subunits, phospholipids and/or surfactants. Thus, the micelles may comprise subunits and detergent, subunits in combination with both phospholipids and detergent, or subunits and phospholipid.

For pharmaceutical compositions which comprise the complexes of the present invention, the dose will vary according to, e.g., the particular complex, the manner of administration, the particular disease being treated and its severity, the overall health and condition of the patient, and the judgment of the prescribing physician. Dosage levels for murine subjects are generally between about 10 μg and about 500 μg. A total dose of between about 50 μg and about 300 μg, is preferred. For instance, in treatments provided over the course of a disease, three 25 μg or 100 μg doses are effective. Total dosages range between about 0.015 and about 15 μg/kg, preferably about 0.15 to about 10 μg/kg.

The pharmaceutical compositions are intended for parenteral, topical, oral or local administration, such as by aerosol or transdermally, for prophylactic and/or therapeutic treatment. The pharmaceutical compositions can be administered in a variety of unit dosage forms depending upon the method of administration. For example, unit dosage forms suitable for oral administration include powder, tablets, pills, and capsules.

Preferably, the pharmaceutical compositions are administered intravenously. Thus, this invention provides compositions for intravenous administration which comprise a solution of the complex dissolved or suspended in an acceptable carrier, preferably an aqueous carrier. A variety of aqueous carriers may be used, e.g., water, buffered water, 0.4% saline, and the like. For instance, phosphate buffered saline (PBS) is particularly suitable for administration of soluble complexes of the present invention. A preferred formulation is PBS containing 0.02% TWEEN-80. These compositions may be sterilized by conventional, well-known sterilization techniques, or may be sterile filtered. The resulting aqueous solutions may be packaged for use as is, or lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The concentration of the complex can vary widely, ie., from less than about 0.05%, usually at or at least about 1% to as much as 10 to 30% by weight and will be selected primarily by fluid volumes, viscosities, etc., in accordance with the particular mode of administration selected. Preferred concentrations for intravenous administration are about 0.02% to about 0.1% or more in PBS.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient.

For aerosol administration, the complexes are preferably supplied in finely divided form along with a surfactant and propellant. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride such as, for example, ethylene glycol, glycerol, erythritol, arabitol, mannitol, sorbitol, the hexitol anhydrides derived from sorbitol, and the polyoxyethylene and polyoxypropylene derivatives of these esters. Mixed esters, such as mixed or natural glycerides may be employed. The surfactant may constitute 0.1%-20% by weight of the composition, preferably 0.25-5%. The balance of the composition is ordinarily propellant. Liquefied propellants are typically gases at ambient conditions, and are condensed under pressure. Among suitable liquefied propellants are the lower alkanes containing up to 5 carbons, such as butane and propane; and preferably fluorinated or fluorochlorinated alkanes. Mixtures of the above may also be employed. In producing the aerosol, a container equipped with a suitable valve is filled with the appropriate propellant, containing the finely divided compounds and surfactant. The ingredients are thus maintained at an elevated pressure until released by action of the valve.

The compositions containing the complexes can be administered for therapeutic, prophylactic, or diagnostic applications. In therapeutic applications, compositions are administered to a patient already suffering from a disease, as described above, in an amount sufficient to cure or at least partially arrest the symptoms of the disease and its complications. An amount adequate to accomplish this is defined as “therapeutically effective dose.” Amounts effective for this use will depend on the severity of the disease and the weight and general state of the patient. As discussed above, this will typically be between about 0.5 mg/kg and about 25 mg/kg, preferably about 3 to about 15 mg/kg.

In prophylactic applications, compositions containing the complexes of the invention are administered to a patient susceptible to or otherwise at risk of a particular disease. Such an amount is defined to be a “prophylactically effective dose.” In this use, the precise amounts again depend on the patient's state of health and weight. The doses will generally be in the ranges set forth above.

In diagnostic applications, compositions containing the appropriate complexes or a cocktail thereof are administered to a patient suspected of having an autoimmune disease state to determine the presence of autoreactive T cells associated with the disease. Alternatively, the efficacy of a particular treatment can be monitored. An amount sufficient to accomplish this is defined to be a “diagnostically effective dose.” In this use, the precise amounts will depend upon the patient's state of health and the like, but generally range from 0.01 to 1000 mg per dose, especially about 10 to about 100 mg per patient.

Kits can also be supplied for therapeutic or diagnostic uses. Thus, the subject composition of the present invention may be provided, usually in a lyophilized form in a container. The complexes, which may be conjugated to a label or toxin, or unconjugated, are included in the kits with buffers, such as Tris, phosphate, carbonate, etc., stabilizers, biocides, inert proteins, e.g., serum albumin, or the like, and a set of instructions for use. Generally, these materials will be present in less than about 5% wt based on the amount of complex, and usually present in total amount of at least about 0.001% wt based again on the protein concentration. Frequently, it will be desirable to include an inert extender or excipient to dilute the active ingredients, where the excipient may be present in from about 1 to 99% wt of the total composition. Where an antibody capable of binding to the complex is employed in an assay, this will usually be present in a separate vial. The antibody is typically conjugated to a label and formulated according to techniques well known in the art.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### VI. EXAMPLES

### Example 1

Determination of IC50 Values for HLA-DR Binding for a Set of Overlapping AChR Peptides

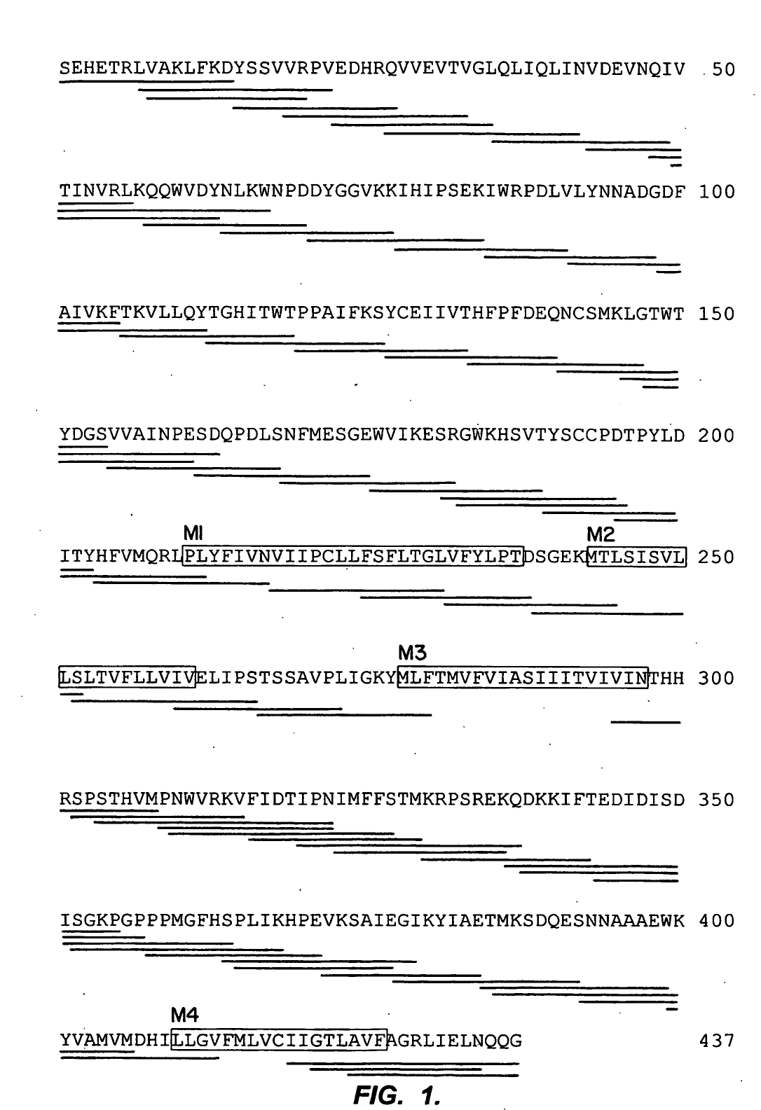

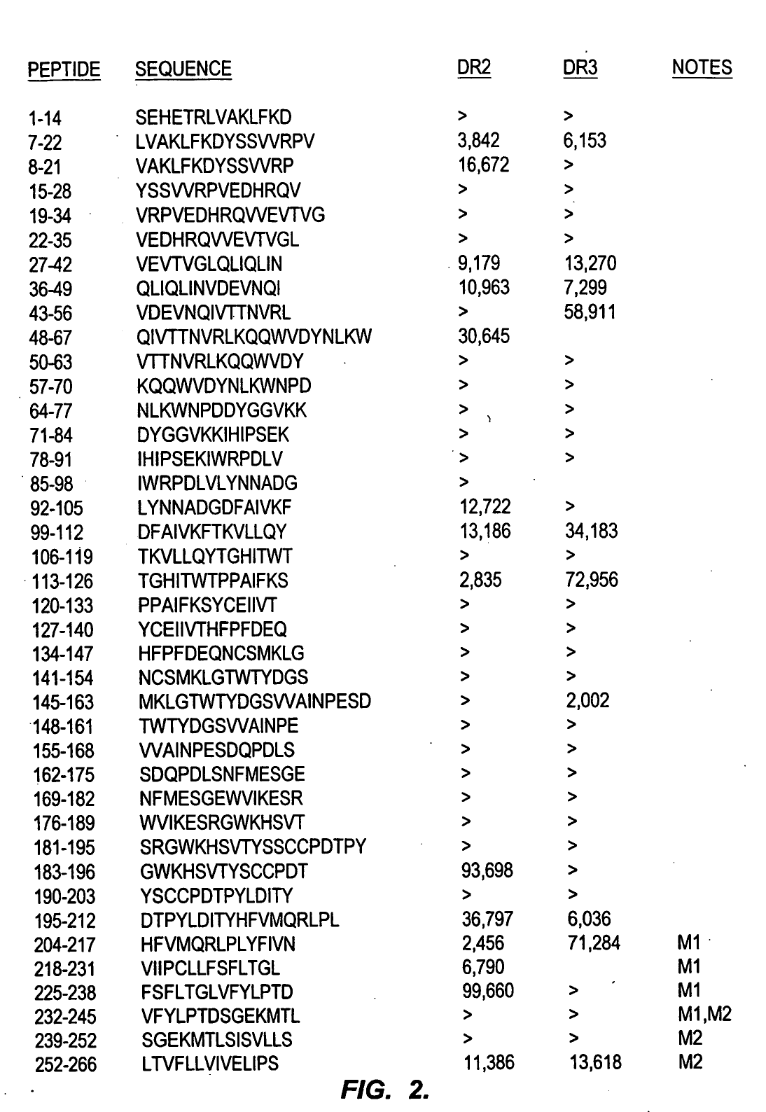

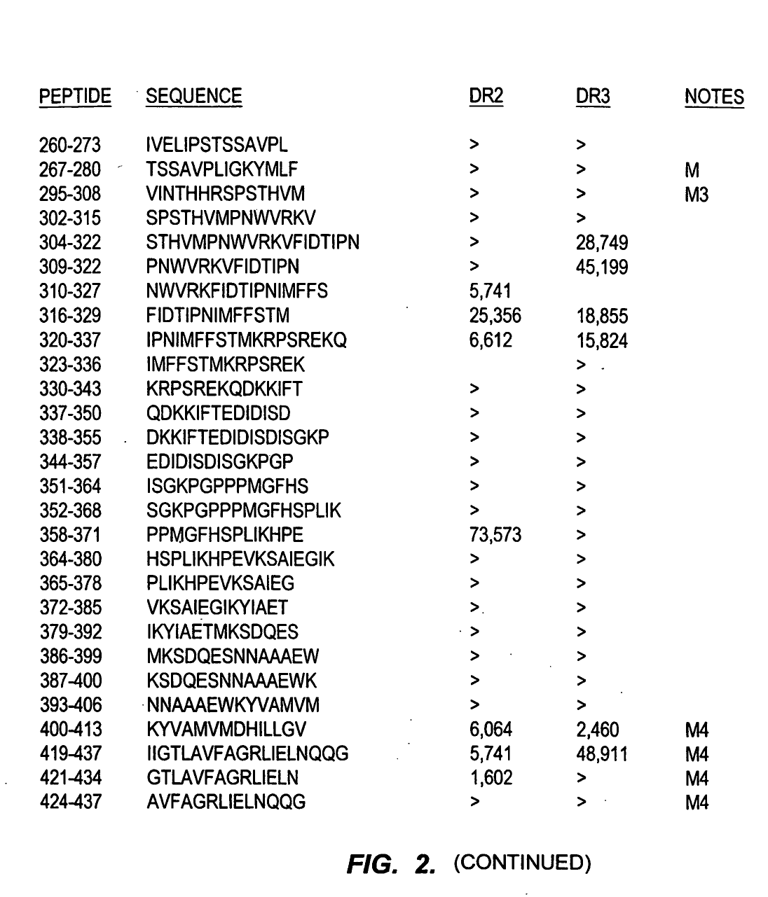

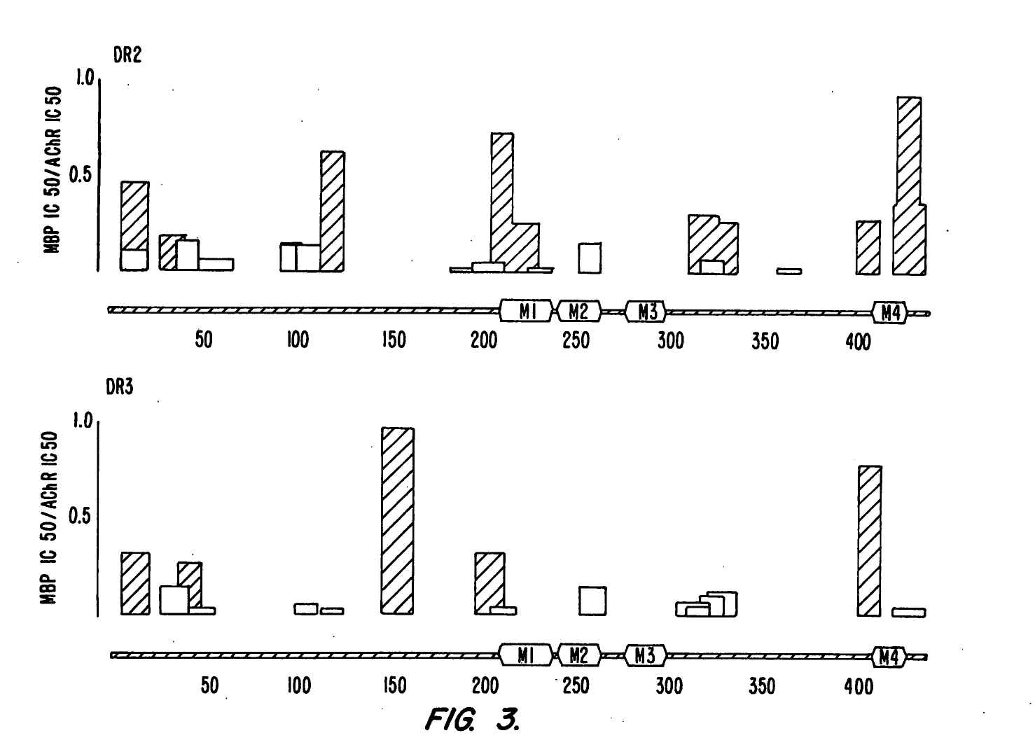

A set of 68 overlapping AChRα peptides (14 mer with 7 amino acids overlapping) was tested by dissociation-enhanced lenthanide fluoroimmunoassay (“DELFIA”) competitive binding assay for their relative affinity to HLA-DR4. FIG. 1 shows the position of overlapping AChR peptides tested in the DELFIA assay of affinity for HLA-DR2 and HLA-DR3. The sequence of the AChRα subunit is shown with each of the transmembrane regions (M1-M4) indicated by a shaded box.) Ten-fold dilutions (preferably 1-100,000 nM) of unlabeled AChRα peptides were co-incubated at pH 5.5 with the biotinylated myelin basic protein peptide MBP 84-102, and the IC₅₀ of binding to solubilized HLA-DR2 (a mix of DRB1*1501 and DRB5*0101) or HLA-DR3 (a mix of DRB1*0301 and DRB3*0101) was measured. Measurement of the IC₅₀ is preferably done by europium-streptavidin dissociation-enhanced lenthanide fluoroimmunoassay (DELFIA) and the IC₅₀ typically calculated by four parameter fit analysis with the software program SOFTmax Pro. FIG. 2 lists the IC₅₀s of each AChRα peptide binding to HLA-DR2 and HLA-DR3. The relative affinity of these peptides is demonstrated in the plot of 1/IC₅₀ in FIG. 3. This graph shows the relative location of the AChRα peptides with the highest relative affinity for HLA-DR1-4. Peptides with high relative affinity for HLA-DR2 include AChRα 7-22, 113-126, 204-217, 310-327, 419-437, and 421-434.

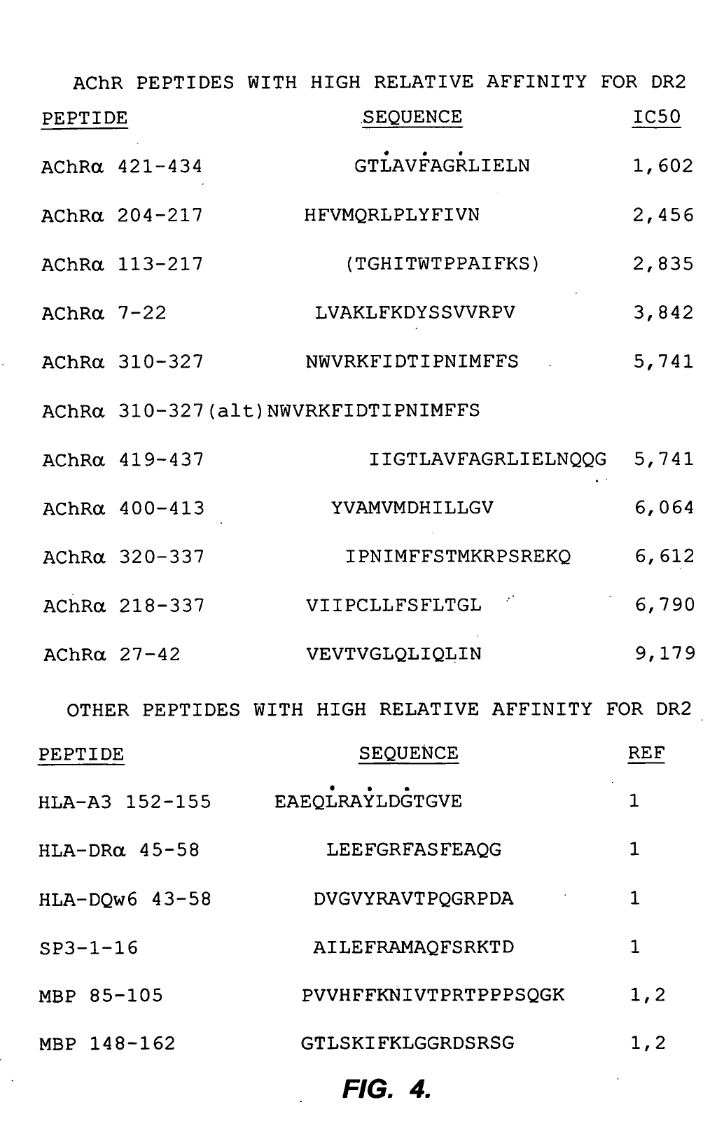

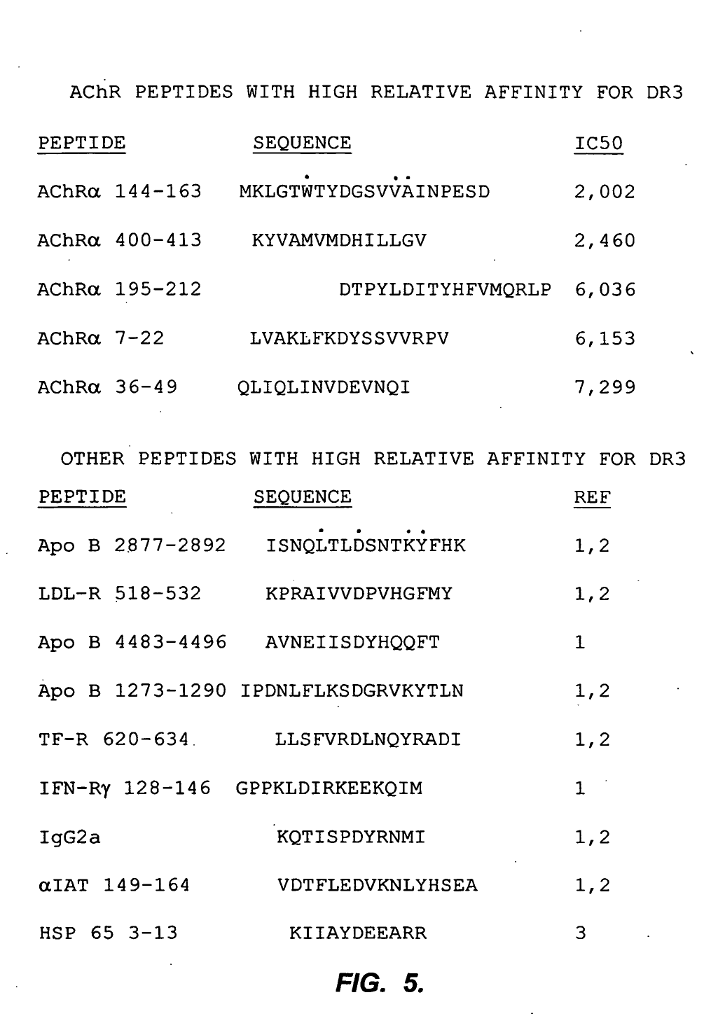

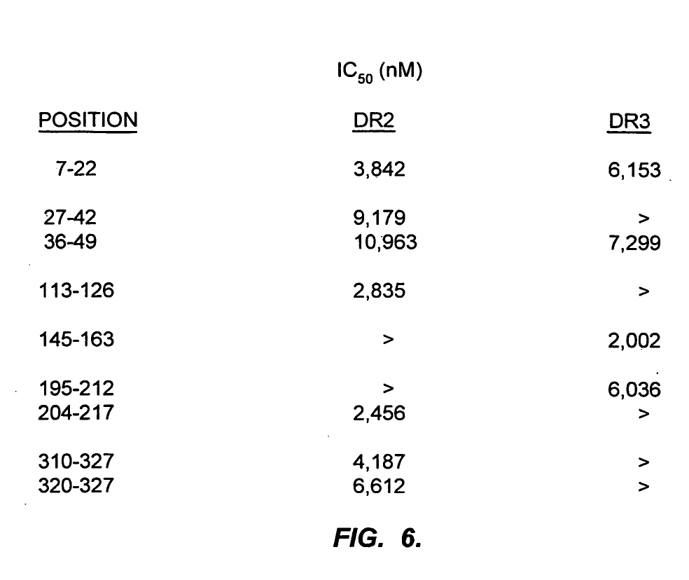

Based on binding studies with phage display libraries, synthethic peptides, and sequencing of eluted peptides several groups have reported binding motifs for DRB1*1501 and DRB5*0101. The peptide fragments showing high affinity for DR2 were found to contain potential the T cell epitopes cited in the literature and a consensus DR2 binding motif. FIG. 4 shows possible alignments of the AChRα peptides with high affinity for HLA-DR2 to match these proposed motifs. Fewer peptides demonstrated high affinity for HLA-DR3, than for HLA-DR2. Those that did exhibit high affinity include 7-22, 36-49, 145-163, 195-212, and 400-413. The DR3 peptide motif is characterized by the nearly universal presence of residue D at position n+3, where n is the anchor residue bound by the DR3 pocket1. FIG. 5 shows possible alignments of the AChRα peptides with high affinity for DR3 to match these proposed motifs. A summary of candidate immunodominant peptides associated with DR2 and DR3 based on IC₅₀≦10,000 nM is listed in FIG. 6.

In addition, the relative affinity for HLA-DR4 of a set of sixty nine synthetic overlapping peptides of AchRα (14 mer with 7 amino acids overlapping) was measured by a europium-based competitive binding assay using solubilized DR4. The concentration of AchR peptides required to inhibit 50% binding of a known DR4 binding peptide was calculated for each peptide. High affinity peptides were identified based on their IC₅₀ value.

Table 2 summarizes the results for five high affinity DR2 binding peptides that were chosen for the ELISPOT studies. These peptides and their IC₅₀ values are given in Table 2.

TABLE 2
AchRα peptide fragments that show lower IC₅₀ values for binding to
HLA-DR2. The lower IC₅₀ value indicates higher binding affinity for
a HLA-DR.

| | |
|---|---|
| AchRα Peptide | IC₅₀ |

| Fragments | DR1 | DR2 | DR3 | DR4 |
|---|---|---|---|---|
| 7-22 | >* | 3,842 | 6,153 | 45,743 |
| 36-49 | 11,185 | 10,963 | 7,299 | 3,626 |
| 204-217 | 1,747 | 2,456 | 71,284 | 31,652 |
| 400-413 | 11,685 | 6,064 | 2,460 | 2,644 |
| 421-4334 | 26,684 | 1,602 | > | > |

IC₅₀ values are much higher than the standard values.

### Example 2

T Cell Reactivity of the AChR Peptides

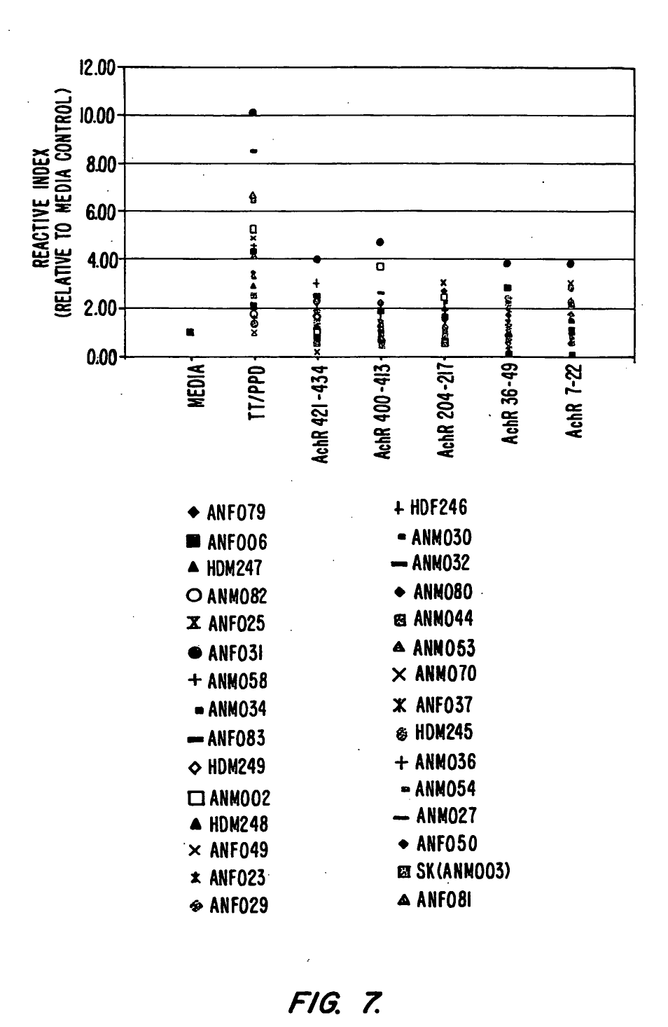

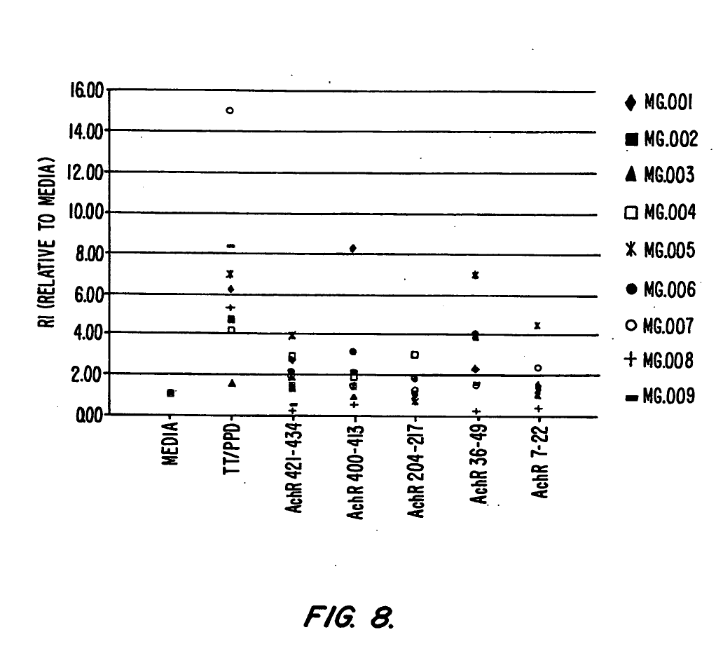

T cell reactivity of the above peptides to patient and normal healthy individual PBMCs was measured by modified ELISPOT assay. PBMCs obtained from 30 normals and 9 MG patients were included in this study. The above peptides along with TT/PPD as positive control and media as negative control were tested for T cell reactivity. Briefly, PBMCs and antigen were incubated for 7 days and an antigen specific clonal expansion was performed on eighth day. Antigen specific cell stimulation profile for each antigen was measured by the secretion of γ-IFN detected as spots using a pair of anti-yIFN antibodies set. The number of spots obtained for each antigen was normalized to the number of spots obtained for the media control. A reactive index, defined as the ratio of number of spots for an antigen to the number of spots for media control, was established for each PBMC sample and each antigen. A plot of reactive index versus antigen used for normal and patient PBMCs is given in FIGS. 7 and 8 respectively.

Comparison of the assay results for the patients and normal individuals indicated no obvious differences in the T cell reactivity to the five AchR epitopes tested. As expected, several MG patients showed reactivity to these AchR epitopes tested but none of them turned out to be unique in terms of high reactivity. It is interesting to note that the normal PBMCs also showed reactivity to these self-antigenic epitopes. This clearly indicates that several other factors, including T cell reactivity to a particular antigen, may be responsible for the initiation of an active disease process. A similar observation has been made in the case of multiple sclerosis and other autoimmune diseases.

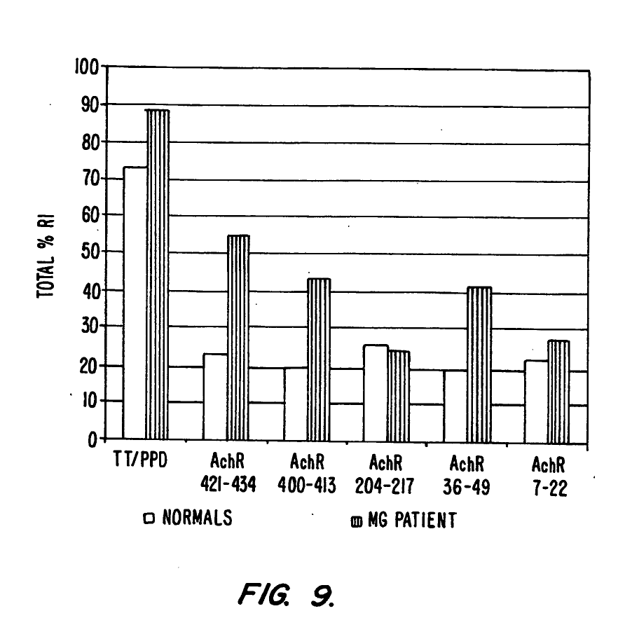

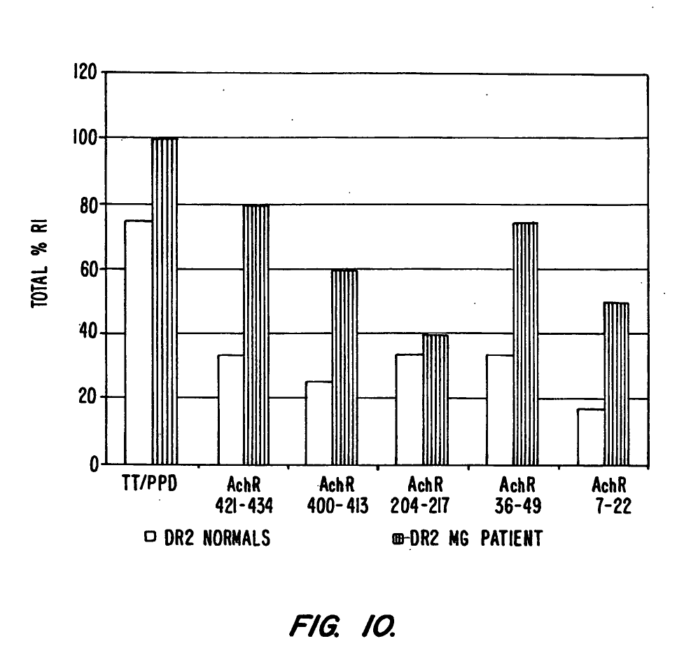

Although the number of patients studied in this project was much smaller than the number of normal healthy individuals, the existence of any preliminary trend in T cell reactivity in patients and normal healthy individuals was tested. The percentage of patients or normal healthy individuals that showed a reactive index above two was compared. Similarly, the existence of a trend in DR2+ patients was also tested. The results of such an analysis of the data are given in FIG. 9 and FIG. 10.

If the HLA type of all the normal healthy individuals and patients is ignored, the per cent of patients that show reactivity to the peptides AChR 421-434, 400-413 and 36-49 is approximately twice the per cent of normal healthy individuals showing the reactivity to these epitopes. This is a further indication that these peptides may be the pathogenic T cell epitopes involved in active disease process. A similar analysis of only DR2+ patients indicated that all the peptides except AchRα 204-217 might be pathogenic T cell epitopes.

Thus, of the 69 AchRα peptides tested, five showed high binding affinity to DR2 (see, Example 1) and four out of these five peptides showed a biased T cell reactivity towards MG patient population as compared to the normal individuals.

From the foregoing, it will be appreciated that, although specific embodiments of the invention have been described herein for the purpose of illustration, various modifications may be made without deviating from the spirit and scope of the invention.

1. A composition comprising an isolated AChR oligopeptide comprising between about 12 and about 20 amino acid residues, wherein the oligopeptide has a sequence substantially similar to a peptide selected from the group consisting of:

| | |
|---|---|
| LVAKLFKDYSSVVRPV, | (SEQ ID NO:2) |
| VEVTVGLQLIQLIN, | (SEQ ID NO:7) |
| TGHITWTPPAIFKS, | (SEQ ID NO:20) |
| HFVMQRLPLYFIVN, | (SEQ ID NO:35) |
| NWVRKVFIDTIPNIMFFS | (SEQ ID NO:47) |
| IPNIMFFSTMKRPSREKQ, | (SEQ ID NO:49) |
| QLIQLINVDEVNQI, | (SEQ ID NO:8) |
| MKLGTWTYDGSVVAINPESD, | (SEQ ID NO:25) |
| and | |
| DTPYLDITYHFVMQRLPL. | (SEQ ID NO:34) |

2. The composition of claim 1, wherein the peptide has a sequence selected from the group consisting of:

| | |
|---|---|
| LVAKLFKDYSSVVRPV, | (SEQ ID NO:2) |
| VEVTVGLQLIQLIN, | (SEQ ID NO:7) |
| TGHITWTPPAIFKS, | (SEQ ID NO:20) |
| HFVMQRLPLYFIVN, | (SEQ ID NO:35) |
| NWVRKVFIDTIPNIMFFS | (SEQ ID NO:47) |
| IPNIMFFSTMKRPSREKQ, | (SEQ ID NO:49) |
| QLIQLINVDEVNQI, | (SEQ ID NO:8) |
| MKLGTWTYDGSVVAINPESD, | (SEQ ID NO:25) |
| and | |
| DTPYLDITYHFVMQRLPL. | (SEQ ID NO:34) |

3. The composition of claim 1, wherein the peptide comprises a D-amino acid or an amino acid mimetic.

4. The composition of claim 1, wherein the oligopeptide is an immunodominant peptide.

5. The composition of claim 1, wherein the peptide is associated with an isolated MHC Class II component having an antigenic binding site, wherein the peptide is associated with the antigenic binding site.

6. The composition of claim 5, wherein the MHC component is an HLA-DR2 molecule.

7. The composition of claim 6, wherein the peptide is selected from the group consisting of:

| | |
|---|---|
| LVAKLFKDYSSVVRPV, | (SEQ ID NO:2) |
| VEVTVGLQLIQLIN, | (SEQ ID NO:7) |
| TGHITWTPPAIFKS, | (SEQ ID NO:20) |
| HFVMQRLPLYFIVN, | (SEQ ID NO:35) |
| NWVRKVFIDTIPNIMFFS | (SEQ ID NO:47) |
| IPNIMFFSTMKRPSREKQ. | (SEQ ID NO:49) |

8. The composition of claim 5, wherein the MHC component is an HLA-DR3 molecule.

9. The composition of claim 8, wherein the peptide is selected from the group consisting of:

| | |
|---|---|
| LVAKLFKDYSSVVRPV, | (SEQ ID NO:2) |
| QLIQLINVDEVNQI, | (SEQ ID NO:8) |
| MKLGTWTYDGSVVAINPESD, | (SEQ ID NO:25) |
| and | |
| DTPYLDITYHFVMQRLPL. | (SEQ ID NO:34) |

10. A composition comprising an antigenic peptide and an isolated MHC component having an antigenic binding site, wherein the antigenic peptide is associated with the antigenic binding site, and wherein the peptide has a sequence substantially similar to a peptide selected from the group consisting of:

| | |
|---|---|
| LVAKLFKDYSSVVRPV, | (SEQ ID NO:2) |
| VEVTVGLQLIQLJN, | (SEQ ID NO:7) |
| TGHITWTPPAIFKS, | (SEQ ID NO:20) |
| HFVMQRLPLYFIVN, | (SEQ ID NO:35) |
| NWVRKVFIDTIPNIMFFS | (SEQ ID NO:47) |
| IPNIMFFSTMKRPSREKQ, | (SEQ ID NO:49) |
| QLIQLINVDEVNQI, | (SEQ ID NO:8) |
| MKLGTWTYDGSVVAINPESD, | (SEQ ID NO:25) |
| and | |
| DTPYLDITYHFVMQRLPL. | (SEQ ID NO:34) |

11. The composition of claim 10, wherein the MHC component is HLA-DR2.

12. The composition of claim 11, wherein the peptide is selected from the group consisting of:

| | |
|---|---|
| LVAKLFKDYSSVVRPV, | (SEQ ID NO:2) |
| VEVTVGLQLIQLIN, | (SEQ ID NO:7) |
| TGHITWTPPAIFKS, | (SEQ ID NO:20) |
| HFVMQRLPLYFIVN, | (SEQ ID NO:35) |
| NWVRKVFIDTIPNIMFFS | (SEQ ID NO:47) |
| IPNIMFFSTMKRPSREKQ. | (SEQ ID NO:49) |

13. The composition of claim 10, wherein the MHC component is HLA-DR3.

14. The composition of claim 13, wherein the peptide is selected from the group consisting of:

| | |
|---|---|
| LVAKLFKDYSSVVRPV, | (SEQ ID NO:2) |
| QLIQLINVDEVNQI, | (SEQ ID NO:8) |
| MKLGTWTYDGSVVAINPESD, | (SEQ ID NO:25) |
| and | |
| DTPYLDITYHFVMQRLPL. | (SEQ ID NO:34) |

15. A pharmaceutical composition comprising a pharmaceutically acceptable carrier and a peptide of claim 1.

16. The pharmaceutical composition of claim 15, wherein the peptide is associated with an isolated MHC class II molecule.

17. The pharmaceutical composition of claim 16, wherein the MHC component is selected from the group consisting of HLA-DR2 and HLA-DR3.

18. A method of treating myasthenia gravis in a patient, the method comprising administering to the patient the pharmaceutical composition of claim 15.

19. A method of treating myasthenia gravis in a patient, the method comprising administering to the patient the pharmaceutical composition of claim 16.

20. A method of treating myasthenia gravis in a patient, the method comprising administering to the patient the pharmaceutical composition of claim 17.

21. A method of inducing non-responsiveness in a target T cell in a mammal, the method comprising administering to the mammal a therapeutically effective dose of a pharmaceutical composition comprising an antigenic peptide and an isolated MHC Class II component having an antigen binding site, wherein the antigenic peptide is associated with the antigen binding site, and wherein the antigenic peptide has a sequence substantially similar to a peptide selected from the group consisting of:

| | |
|---|---|
| LVAKLFKDYSSVVRPV, | (SEQ ID NO:2) |
| VEVTVGLQLIQLIN, | (SEQ ID NO:7) |
| TGHITWTPPAIFKS, | (SEQ ID NO:20) |
| HFVMQRLPLYFIVN, | (SEQ ID NO:35) |
| NWVRKVFIDTIPNIMFFS | (SEQ ID NO:47) |
| IPNIMFFSTMKRPSREKQ, | (SEQ ID NO:49) |
| QLIQLINVDEVNQI, | (SEQ ID NO:8) |
| MKLGTWTYDGSVVAINPESD, | (SEQ ID NO:25) |
| and | |
| DTPYLDITYHFVMQRLPL. | (SEQ ID NO:34) |

22. The method of claim 21, wherein the antigenic peptide has a sequence selected from the group consisting of:

| | |
|---|---|
| LVAKLFKDYSSVVRPV, | (SEQ ID NO:2) |
| VEVTVGLQLIQLIN, | (SEQ ID NO:7) |
| TGHITWTPPAIFKS, | (SEQ ID NO:20) |
| HFVMQRLPLYFIVN, | (SEQ ID NO:35) |
| NWVRKVFIDTIPNIMFFS | (SEQ ID NO:47) |
| IPNIMFFSTMKRPSREKQ, | (SEQ ID NO:49) |
| QLIQLINVDEVNQI, | (SEQ ID NO:8) |
| MKLGTWTYDGSVVAINPESD, | (SEQ ID NO:25) |
| and | |
| DTPYLDITYHFVMQRLPL. | (SEQ ID NO:34) |

23. The method of claim 21, wherein the MHC component is HLA-DR2.

24. The method of claim 21, wherein the MHC component is HLA-DR3.

25. The method of claim 21, wherein the pharmaceutical composition is administered intravenously.


SEQUENCE LISTING (86 sequences)

SEQ ID NO: 1 - protein, 14 aa, Artificial Sequence
  acetylcholine receptor (AchR) alpha peptide 1-14
SEHETRLVAKLFKD

SEQ ID NO: 2 - protein, 16 aa, Artificial Sequence
  acetylcholine receptor (AchR) alpha peptide 7-22
LVAKLFKDYSSVVRPV

SEQ ID NO: 3 - protein, 14 aa, Artificial Sequence
  acetylcholine receptor (AchR) alpha peptide 8-21
VAKLFKDYSSVVRP

SEQ ID NO: 4 - protein, 14 aa, Artificial Sequence
  acetylcholine receptor (AchR) alpha peptide 15-28
YSSVVRPVEDHRQV

SEQ ID NO: 5 - protein, 16 aa, Artificial Sequence
  acetylcholine receptor (AchR) alpha peptide 19-34
VRPVEDHRQVVEVTVG

SEQ ID NO: 6 - protein, 14 aa, Artificial Sequence
  acetylcholine receptor (AchR) alpha peptide 22-35
VEDHRQVVEVTVGL

SEQ ID NO: 7 - protein, 14 aa, Artificial Sequence
  acetylcholine receptor (AchR) alpha peptide 22-42
VEVTVGLQLIQLIN

SEQ ID NO: 8 - protein, 14 aa, Artificial Sequence
  acetylcholine receptor (AchR) alpha peptide 36-49
QLIQLINVDEVNQI

SEQ ID NO: 9 - protein, 14 aa, Artificial Sequence
  acetylcholine receptor (AchR) alpha peptide 43-56
VDEVNQIVTTNVRL

SEQ ID NO: 10 - protein, 20 aa, Artificial Sequence
  acetylcholine receptor (AchR) alpha peptide 48-67
QIVTTNVRLKQQWVDYNLKW

SEQ ID NO: 11 - protein, 14 aa, Artificial Sequence
  acetylcholine receptor (AchR) alpha peptide 50-63
VTTNVRLKQQWVDY

SEQ ID NO: 12 - protein, 14 aa, Artificial Sequence
  acetylcholine receptor (AchR) alpha peptide 57-70
KQQWVDYNLKWNPD

SEQ ID NO: 13 - protein, 14 aa, Artificial Sequence
  acetylcholine receptor (AchR) alpha peptide 64-77
NLKWNPDDYGGVKK

SEQ ID NO: 14 - protein, 14 aa, Artificial Sequence
  acetylcholine receptor (AchR) alpha peptide 71-84
DYGGVKKIHIPSEK

SEQ ID NO: 15 - protein, 14 aa, Artificial Sequence
  acetylcholine receptor (AchR) alpha peptide 78-91
IHIPSEKIWRPDLV

SEQ ID NO: 16 - protein, 14 aa, Artificial Sequence
  acetylcholine receptor (AchR) alpha peptide 85-98
IWRPDLVLYNNADG

SEQ ID NO: 17 - protein, 14 aa, Artificial Sequence
  acetylcholine receptor (AchR) alpha peptide 92-105
LYNNADGDFAIVKF

SEQ ID NO: 18 - protein, 14 aa, Artificial Sequence
  acetylcholine receptor (AchR) alpha peptide 99-112
DFAIVKFTKVLLQY

SEQ ID NO: 19 - protein, 14 aa, Artificial Sequence
  acetylcholine receptor (AchR) alpha peptide 106-119
TKVLLQYTGHITWT

SEQ ID NO: 20 - protein, 14 aa, Artificial Sequence
  acetylcholine receptor (AchR) alpha peptide 113-126
TGHITWTPPAIFKS

SEQ ID NO: 21 - protein, 14 aa, Artificial Sequence
  acetylcholine receptor (AchR) alpha peptide 120-133
PPAIFKSYCEIIVT

SEQ ID NO: 22 - protein, 14 aa, Artificial Sequence
  acetylcholine receptor (AchR) alpha peptide 127-140
YCEIIVTHFPFDEQ

SEQ ID NO: 23 - protein, 14 aa, Artificial Sequence
  acetylcholine receptor (AchR) alpha peptide 134-147
HFPFDEQNCSMKLG

SEQ ID NO: 24 - protein, 14 aa, Artificial Sequence
  acetylcholine receptor (AchR) alpha peptide 141-154
NCSMKLGTWTYDGS

SEQ ID NO: 25 - protein, 20 aa, Artificial Sequence
  acetylcholine receptor (AchR) alpha peptide 145-163
MKLGTWTYDGSVVAINPESD

SEQ ID NO: 26 - protein, 14 aa, Artificial Sequence
  acetylcholine receptor (AchR) alpha peptide 148-161
TWTYDGSVVAINPE

SEQ ID NO: 27 - protein, 14 aa, Artificial Sequence
  acetylcholine receptor (AchR) alpha peptide 155-168
VVAINPESDQPDLS

SEQ ID NO: 28 - protein, 14 aa, Artificial Sequence
  acetylcholine receptor (AchR) alpha peptide 162-175
SDQPDLSNFMESGE

SEQ ID NO: 29 - protein, 14 aa, Artificial Sequence
  acetylcholine receptor (AchR) alpha peptide 169-182
NFMESGEWVIKESR

SEQ ID NO: 30 - protein, 14 aa, Artificial Sequence
  acetylcholine receptor (AchR) alpha peptide 176-189
WVIKESRGWKHSVT

SEQ ID NO: 31 - protein, 18 aa, Artificial Sequence
  acetylcholine receptor (AchR) alpha peptide 181-195
SRGWKHSVTYSCCPDTPY

SEQ ID NO: 32 - protein, 14 aa, Artificial Sequence
  acetylcholine receptor (AchR) alpha peptide 183-196
GWKHSVTYSCCPDT

SEQ ID NO: 33 - protein, 14 aa, Artificial Sequence
  acetylcholine receptor (AchR) alpha peptide 190-203
YSCCPDTPYLDITY

SEQ ID NO: 34 - protein, 18 aa, Artificial Sequence
  acetylcholine receptor (AchR) alpha peptide 195-212
DTPYLDITYHFVMQRLPL

SEQ ID NO: 35 - protein, 14 aa, Artificial Sequence
  acetylcholine receptor (AchR) alpha peptide 204-217
HFVMQRLPLYFIVN

SEQ ID NO: 36 - protein, 14 aa, Artificial Sequence
  acetylcholine receptor (AchR) alpha peptide 218-231
VIIPCLLFSFLTGL

SEQ ID NO: 37 - protein, 14 aa, Artificial Sequence
  acetylcholine receptor (AchR) alpha peptide 225-238
FSFLTGLVFYLPTD

SEQ ID NO: 38 - protein, 14 aa, Artificial Sequence
  acetylcholine receptor (AchR) alpha peptide 232-245
VFYLPTDSGEKMTL

SEQ ID NO: 39 - protein, 14 aa, Artificial Sequence
  acetylcholine receptor (AchR) alpha peptide 239-252
SGEKMTLSISVLLS

SEQ ID NO: 40 - protein, 14 aa, Artificial Sequence
  acetylcholine receptor (AchR) alpha peptide 252-266
LTVFLLVIVELIPS

SEQ ID NO: 41 - protein, 14 aa, Artificial Sequence
  acetylcholine receptor (AchR) alpha peptide 260-273
IVELIPSTSSAVPL

SEQ ID NO: 42 - protein, 14 aa, Artificial Sequence
  acetylcholine receptor (AchR) alpha peptide 267-280
TSSAVPLIGKYMLF

SEQ ID NO: 43 - protein, 14 aa, Artificial Sequence
  acetylcholine receptor (AchR) alpha peptide 295-308
VINTHHRSPSTHVM

SEQ ID NO: 44 - protein, 14 aa, Artificial Sequence
  acetylcholine receptor (AchR) alpha peptide 302-315
SPSTHVMPNWVRKV

SEQ ID NO: 45 - protein, 19 aa, Artificial Sequence
  acetylcholine receptor (AchR) alpha peptide 304-322
STHVMPNWVRKVFIDTIPN

SEQ ID NO: 46 - protein, 14 aa, Artificial Sequence
  acetylcholine receptor (AchR) alpha peptide 309-322
PNWVRKVFIDTIPN

SEQ ID NO: 47 - protein, 18 aa, Artificial Sequence
  acetylcholine receptor (AchR) alpha peptide 310-327
NWVRKVFIDTIPNIMFFS

SEQ ID NO: 48 - protein, 14 aa, Artificial Sequence
  acetylcholine receptor (AchR) alpha peptide 316-329
FIDTIPNIMFFSTM

SEQ ID NO: 49 - protein, 18 aa, Artificial Sequence
  acetylcholine receptor (AchR) alpha peptide 320-337
IPNIMFFSTMKRPSREKQ

SEQ ID NO: 50 - protein, 14 aa, Artificial Sequence
  acetylcholine receptor (AchR) alpha peptide 323-336
IMFFSTMKRPSREK

SEQ ID NO: 51 - protein, 14 aa, Artificial Sequence
  acetylcholine receptor (AchR) alpha peptide 330-343
KRPSREKQDKKIFT

SEQ ID NO: 52 - protein, 14 aa, Artificial Sequence
  acetylcholine receptor (AchR) alpha peptide 337-350
QDKKIFTEDIDISD

SEQ ID NO: 53 - protein, 18 aa, Artificial Sequence
  acetylcholine receptor (AchR) alpha peptide 338-355
DKKIFTEDIDISDISGKP

SEQ ID NO: 54 - protein, 14 aa, Artificial Sequence
  acetylcholine receptor (AchR) alpha peptide 344-357
EDIDISDISGKPGP

SEQ ID NO: 55 - protein, 14 aa, Artificial Sequence
  acetylcholine receptor (AchR) alpha peptide 351-364
ISGKPGPPPMGFHS

SEQ ID NO: 56 - protein, 17 aa, Artificial Sequence
  acetylcholine receptor (AchR) alpha peptide 352-368
SGKPGPPPMGFHSPLIK

SEQ ID NO: 57 - protein, 14 aa, Artificial Sequence
  acetylcholine receptor (AchR) alpha peptide 358-371
PPMGFHSPLIKHPE

SEQ ID NO: 58 - protein, 18 aa, Artificial Sequence
  acetylcholine receptor (AchR) alpha peptide 364-380
HSPLIKHPEVKSAIEGIK

SEQ ID NO: 59 - protein, 14 aa, Artificial Sequence
  acetylcholine receptor (AchR) alpha peptide 365-378
PLIKHPEVKSAIEG

SEQ ID NO: 60 - protein, 14 aa, Artificial Sequence
  acetylcholine receptor (AchR) alpha peptide 372-385
VKSAIEGIKYIAET

SEQ ID NO: 61 - protein, 14 aa, Artificial Sequence
  acetylcholine receptor (AchR) alpha peptide 379-392
IKYIAETMKSDQES

SEQ ID NO: 62 - protein, 14 aa, Artificial Sequence
  acetylcholine receptor (AchR) alpha peptide 386-399
MKSDQESNNAAAEW

SEQ ID NO: 63 - protein, 14 aa, Artificial Sequence
  acetylcholine receptor (AchR) alpha peptide 387-400
KSDQESNNAAAEWK

SEQ ID NO: 64 - protein, 14 aa, Artificial Sequence
  acetylcholine receptor (AchR) alpha peptide 393-406
NNAAAEWKYVAMVM

SEQ ID NO: 65 - protein, 14 aa, Artificial Sequence
  acetylcholine receptor (AchR) alpha peptide 400-413
KYVAMVMDHILLGV

SEQ ID NO: 66 - protein, 19 aa, Artificial Sequence
  acetylcholine receptor (AchR) alpha peptide 419-437
IIGTLAVFAGRLIELNQQG

SEQ ID NO: 67 - protein, 14 aa, Artificial Sequence
  acetylcholine receptor (AchR) alpha peptide 421-434
GTLAVFAGRLIELN

SEQ ID NO: 68 - protein, 14 aa, Artificial Sequence
  acetylcholine receptor (AchR) alpha peptide 424-437
AVFAGRLIELNQQG

SEQ ID NO: 69 - protein, 437 aa, Homo sapiens
  acetylcholine receptor (AchR) alpha
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLPLYFIVNVIIPCLLFSFLTGLVFYLPTDSGEKMTLSISVLLSLTVFLLVIVELIPSTSSAVPLIGKYMLFTMVFVIASIIITVIVINTHHRSPSTHVMPNWVRKVFIDTIPNIMFFSTMKRPSREKQDKKIFTEDIDISDISGKPGPPPMGFHSPLIKHPEVKSAIEGIKYIAETMKSDQESNNAAAEWKYVAMVMDHILLGVFMLVCIIGTLAVFAGRLIELNQQG

SEQ ID NO: 70 - protein, 15 aa, Artificial Sequence
  HLA-A3 peptide 152-166
EAEQLRAYLDGTGVE

SEQ ID NO: 71 - protein, 14 aa, Artificial Sequence
  HLA-DRalpha peptide 45-58
LEEFGRFASFEAQG

SEQ ID NO: 72 - protein, 16 aa, Artificial Sequence
  HLA-DQw6 peptide 43-58
DVGVYRAVTPQGRPDA

SEQ ID NO: 73 - protein, 16 aa, Artificial Sequence
  SP3 peptide 1-16
AILEFRAMAQFSRKTD

SEQ ID NO: 74 - protein, 21 aa, Artificial Sequence
  MBP peptide 85-105
PVVHFFKNIVTPRTPPPSQGK

SEQ ID NO: 75 - protein, 17 aa, Artificial Sequence
  MBP peptide 148-162
GTLSKIFKLGGRDSRSG

SEQ ID NO: 76 - protein, 16 aa, Artificial Sequence
  Apo B peptide 2877-2892
ISNQLTLDSNTKYFHK

SEQ ID NO: 77 - protein, 15 aa, Artificial Sequence
  LDL-R peptide 518-532
KPRAIVVDPVHGFMY

SEQ ID NO: 78 - protein, 14 aa, Artificial Sequence
  Apo B peptide 4483-4496
ANVEIISDYHQQFT

SEQ ID NO: 79 - protein, 18 aa, Artificial Sequence
  Apo B peptide 1273-1290
IPDNLFLKSDGRVKYTLN

SEQ ID NO: 80 - protein, 15 aa, Artificial Sequence
  TF-R peptide 620-634
LLSFVRDLNQYRADI

SEQ ID NO: 81 - protein, 15 aa, Artificial Sequence
  IFN-Rgamma peptide 128-146
GPPKLDIRKEEKQIM

SEQ ID NO: 82 - protein, 12 aa, Artificial Sequence
  IgG2a peptide
KQTISPDYRNMI

SEQ ID NO: 83 - protein, 16 aa, Artificial Sequence
  alphaIAT peptide 149-164
VDTFLEDVKNLYHSEA

SEQ ID NO: 84 - protein, 11 aa, Artificial Sequence
  HSP 65 peptide 3-13
KIIAYDEEARR

SEQ ID NO: 85 - protein, 13 aa, Artificial Sequence
  acetylcholine receptor (AchR) alpha peptide 401-413
YVAMVMDHILLGV

SEQ ID NO: 86 - protein, 17 aa, Artificial Sequence
  acetylcholine receptor (AchR) alpha peptide 195-212
DTPYLDITYHFVMQRLP